(19) United States
(12) Patent Application Publication
(10) Pub. No.: US 2010/0215763 A1
(43) Pub. Date: Aug. 26, 2010

(54) Method of using the body's alternative cellular energy pigments (ACE-pigments) in the therapy of diseases

(76) Inventor: William John Martin, South Pasadena, CA (US)

Correspondence Address: William John Martin, 1634 Spruce Street, South Pasadena, CA, 91030, US

(21) Appl. No.: 12/378,934

(22) Filed: Feb. 20, 2009

(51) Int. Cl. A61K 35/24

(52) U.S. Cl. 424/537

(57) ABSTRACT

Alternative cellular energy pigments (ACE-pigments) are over-produced in response to various types of viral infections and to other pathological conditions. ACE pigments accumulate at the sites of disease and also in other regions of the body, including the oral cavity. ACE pigments are considered to play a significant role in the body's healing response to both local and system diseases. They appear to exist in a relatively inactive form in which they can fluorescence under ultraviolet (UV) light illumination, especially if stained with a suitable dye, such as neutral red. The present invention describes a method of using ACE pigments collected from the saliva (spittle) of patients for the purpose of activating the ACE pathway in a child with autism and in an adult with an active skin lesion caused by herpes simplex virus. The saliva containing the collected ACE pigments is placed onto a suitable material, such as a paper towel, stained with neutral red dye and induced to fluoresce using UV illumination. The fluorescing mixture is placed over various areas of the body of a child with autism and/or directly placed over the herpes simplex virus induced skin lesions, and the UV illumination continued. This process will partially activate the ACE pathway in the patient with resulting clinical benefit.

### CROSS REFERENCE TO CO-PENDING PATENT APPLICATION

Methods for Collecting and Using the Body's Alternative Cellular Energy Pigments (ACE-pigments) in the Therapy of Diseases. Submitted Feb. 2, 2009

Methods for Detection of Ultraviolet Reactive Alternative Cellular Energy Pigments (ACE-Pigments). William John Martin Submitted Dec. 24, 2007.

Method of Assessing and of Activating the Alternative Cellular Energy (ACE) Pathway in the Therapy of Diseases. William John Martin Submitted Jan. 17, 2008

Enerceutical Mediated Activation of the Alternative Cellular Energy (ACE) Pathway in the Therapy of Diseases Submitted May 8, 2008

Method of Activating the Alternative Cellular Energy (ACE) Pathway Using Hydrogen. Submitted Jul. 9, 2008

### CROSS REFERENCE TO PREVIOUSLY SUBMITTED BUT NOW ABANDONED PATENT APPLICATIONS

Ser. No. 10/044,683. Therapy of stealth virus associated cancers and other conditions using light. William John Martin. (Abandoned)
Ser. No. 10/047,313. Therapy of stealth virus associated cancers and other conditions using medium chain triglycerides. William John Martin. (Abandoned)
Ser. No. 10/050,232. Diagnosing and monitoring the therapy of stealth virus infections based on the detection of auto-fluorescent material in hair. William John Martin. (Abandoned)
Ser. No. 10/058,480. Therapy of stealth virus associated cancers and other conditions using magnetic energy. William John Martin. (Abandoned)
Ser. No. 10/164,258 Energy supportive therapy of stealth virus associated diseases. William John Martin. (Abandoned)
Ser. No. 10/174,466 Sound therapy of stealth virus associated diseases. William John Martin. (Abandoned)
Ser. No. 10/192,936 ACE-Pigments and humic acids as energy sources. William John Martin. (Abandoned)
Submitted: Ser. No. 10/______ Methods for Collection of Alternative Cellular Energy Pigments (ACE-pigments). William John Martin. (Abandoned)
Submitted: Ser. No. 10/______ Methods for Elimination of Toxic Alternative Cellular Energy Pigments (ACE-pigments) and for Their Replacement Using Activated Humates, Including Humic and Fulvic Acids. William John Martin. (Abandoned)

### UNITED STATES PATENTS (AWARDED)

U.S. Pat. No. 5,985,546 Stealth virus detection in the chronic fatigue syndrome. William John Martin
U.S. Pat. No. 5,891,468 Stealth virus detection in the chronic fatigue syndrome. William John Martin
U.S. Pat. No. 5,753,488 Isolated stealth viruses and related vaccines. William John Martin
U.S. Pat. No. 5,703,221 Stealth virus nucleic acids and related methods. William John Martin
U.S. Pat. No. 6,368,637. Method and composition for topical treatment of viral lesions. Jon Stoneburner

### PCT (PATENT COOPERATION TREATY)

WO 92/20797 Stealth virus detection in the chronic fatigue syndrome. William John Martin
WO 99/34019 Stealth virus nucleic acids and related methods. William John Martin
WO 99/60101 Stealth viruses and related vaccines. William John Martin

### REFERENCES TO PUBLISHED AND TO SUBMITTED ARTICLES ALTERNATIVE CELLULAR ENERGY PIGMENTS (ACE-PIGMENTS):

1 Martin W J. Alternative cellular energy pigments mistaken for parasitic skin infestations. Exp. Mol. Path 78: 212-214, 2005.

2 Martin W J. Alternative cellular energy pigments from bacteria of stealth virus infected individuals. Exp. Mol. Path 78: 217-217, 2005.

3 Martin W J. Progressive Medicine. Exp Mol Path 78: 218-220, 2005.

4 Martin W J, Stoneburner J. Symptomatic relief of herpetic skin lesions utilizing an energy based approach to healing. Exp. Mol. Path 78: 131-4, 2005.

5 Martin W J. Etheric Biology. Exp Mol Path 78: 221-227, 2005.

6 Martin W J. Stealth Virus Culture Pigments: A Potential Source of Cellular Energy. Exp. Mol. Pathol. 74: 210-223, 2003.

7 Martin W J. Complex intracellular inclusions in the brain of a child with a stealth virus encephalopathy. Exp. Mol. Pathol. 74: 179-209, 2003.

8 Martin W J. Photons and phonons: Theoretical aspects of biophysics and potential therapeutic applications. Proceeding of Neural Therapy Workshop on Sound and Light Therapy, Seattle, Wash., Feb. 21-23, 2003.

9 Martin W J. Activation of the Alternative Cellular Energy (ACE) Pathway as Natural Therapy for Herpes Simplex and Herpes Zoster Virus Infections. Submitted electronically to Journal of Infectious Diseases on 3/198/2008 Article rejected for publication. Revised article in progress.

10 Martin W J. Activation of the Alternative Cellular Energy (ACE) Pathway as Natural Therapy for Patients with Autism. Submitted electronically to Journal of Autism and Disability Disorders on Jun. 24, 2008. Article rejected for publication. Revised article in progress.

### STEALTH ADAPTED VIRUSES

1 Martin W J Chronic fatigue syndrome among physicians. A potential result of occupational exposure to stealth viruses. Explore 2001; 10: 7-10.

2 Martin W J. Stealth Viruses. Explore 2001; 10: 17-19.

3 Durie G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J., Anderson D. Stealth virus epidemic in the Mohave Valley: severe vacuolating encephalopathy in a child presenting with a behavioral disorder. Exp Mol Pathol. 1999; 66:19-30.

6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin W J. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J., Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome. Pathobiology. 1996; 64:9-17.

15 Martin W J. Severe stealth virus encephalopathy following chronic-fatigue-syndrome-like illness: clinical and histopathological features. Pathobiology. 1996; 64:1-8.

≠Martin W J. Stealth virus isolated from an autistic child. J Autism Dev Disord. 1995; 25:223-4.

17 Gollard R P, Mayr A., Rice D A, Martin W J. Herpesvirus-related sequences in salivary gland tumors. J Exp Clin Cancer Res., 1996; 15: 1-4.

18 Martin W J., Glass R T. Acute encephalopathy induced in cats with a stealth virus isolated from a patient with chronic fatigue syndrome. Pathobiology. 1995; 63:115-8.

19 Martin W J, et al. African green monkey origin of the atypical cytopathic ‘stealth virus’ isolated from a patient with chronic fatigue syndrome. Clin Diag Virol 1995: 4: 93-103.

20 Martin W J. Stealth viruses as neuropathogens. CAP Today. 1994; 8: 67-70.

21 Martin W J. et al. Cytomegalovirus-related sequence in an atypical cytopathic virus repeatedly isolated from a patient with chronic fatigue syndrome. Am J Pathol. 1994; 145: 440-51.

### STATEMENT REGARDING FEDERALLY SPONSORED RESEARCH OR DEVELOPMENT

No Federal funding was received in support of the research covered in this patent application.

### REFERENCE TO SEQUENCE LISTING, A TABLE, OR A COMPUTER PROGRAM LISTING COMPACT DICK APPENDIX

None provided.

### BACKGROUND

With the exception of chlorophyll-containing plants and bacteria, it is widely believed that virtually all of the energy produced by living organisms is derived from the catabolic (metabolic breakdown) of food. The applicant has proven that an additional source of cellular energy is available to living cells. It is derived through the conversion of physical energies, including electromagnetic radiation, magnetic fields, sound, etc., to biological energy that can be used for biosynthetic reactions and other cellular functions. This energy conversion (transduction) is mediated by materials that the applicant has termed “alternative cellular energy pigments” (ACE-pigments). These pigments were identified as intracellular inclusions that can lead to extra-cellular particles in the liquid medium of cultured cells infected with cell damaging (cytopathic) stealth adapted viruses. The viruses were termed “stealth” or “stealth-adapted” because they do not evoke an inflammatory reaction within the infected individuals. They are molecularly heterogeneous, although some are unequivocally derived from herpes viruses, including African green monkey simian cytomegalovirus (SCMV). Stealth adapted viruses have been cultured from patients with a wide range of neuropsychiatric and other illnesses.

The pigmented structures that develop in cultures of stealth adapted viruses are conglomerates of smaller microscopic and sub-microscopic particles. They can also take the form of long threads and ribbon-like structures. ACE-pigments show various properties indicative of their capacity to absorb light energy (photons). For example, the particles will typically display microscopic movements within a fluid environment, which far exceed that explainable as simple Brownian motility. These movements can be shown to be at least in part in response to the illumination delivered by the microscope. The larger particles can occasionally display slow oscillatory (back and forth) movements in response to light illumination. The smaller particles and some of the fibers and threads will commonly fluoresce slightly when exposed to ultraviolet (UV) light. Much of the emitted fluorescence is in the green to yellow range, but occasionally it is strikingly red. Even green visible light can provoke red emission from some particles. The induced fluorescence of the particles can be greatly enhanced in the presence of certain dyes, including neutral red, a tricyclic non-fluorescent dye. The particles can also differentially alter the emitted fluorescence of known UV fluorescent dyes such as acridine orange. As expected, stealth virus infected cells stained with either neutral red or acridine orange contains finely dispersed material that displays multi-colored (green, yellow and red) fluorescence, which is not seen in uninfected cells. Similar multicolor UV particulate fluorescence can often be seen in the cytoplasm of neutral red stained polymorphonuclear cells obtained from both known and presumptively stealth adapted virus infected patients.

Fluorescing materials, either alone or after mixing with neutral red dye, can also be collected directly from the skin or from perspiration of various patients. Methods to enhance perspiration such as the use of heat can increase the secretion of ACE pigments, as can the taking of a hot bath. Another collection method is to have a perspiring patient sleep without clothing between clean sheets from which the particles can be retrieved the next morning. Hair samples can have attached fluorescing materials and/or display intrinsic fluorescent properties. Fluorescing materials, especially after mixing with a small amount of neutral red dye, can also be retrieved from spittle (spit) and from the buccal, tongue and palatal surfaces of the mouths of various patients. Discrete particles can occasionally be recognized by the evoked fluorescence under UV microscopy, especially when viewed in liquid medium since this method can also reveal the characteristic non-Brownian movements of light activated particles.

Larger amounts of ACE pigments are commonly present the vicinity of skin lesions caused by Herpes simplex virus (HSV) and Herpes zoster virus (HZV) (hereafter occasionally lumped together as herpetic skin lesions). They also occur in association with warts caused by human papillomavirus (HPV) and are presumably part of the body's response to many other chronic infectious agents, including hepatitis viruses and HIV. Accumulations of ACE pigments have also been detected in association with other chronic skin conditions including squamous cell carcinomas, seborrheic keratosis and psoriasis. One method for detecting ACE pigments in skin lesions is by rubbing the lesion with a Q-tip or simple gauze swab, staining with a dilute solution of neutral red (typically 0.1 to 1 mg/ml) and observing for UV inducible yellowish fluorescence. Absorbent paper can also be used to collect ACE pigments from skin lesions, as well as from broader areas of the skin, hair, mouth, etc.

ACE pigments were identified as potential therapeutic agents when it was shown that their appearance in stealth adapted viral cultures correlated with a lessening of the cytopathic effect (CPE) caused by the viruses. Thus, for example, removal of the ACE-pigments from the cultures resulted in a rapid reactivation of the CPE that could be very easily prevented by the re-addition of isolated ACE pigments to the fresh re-feeding culture medium. Rapid cellular repair is achieved even though the added ACE pigments are essentially only present in the supernatants, suggesting a biophysical effect occurring over a distance, rather than a biochemical process that would require penetration of the particles into the cells.

The healing properties of ACE pigments was further suggested by the expedited healing that occurs when neutral red is applied to active HSV and HZV skin lesions followed by UV illumination. Prior to the discovery of ACE pigments, the success of this procedure that dates back to the 1970's was incorrectly attributed to a direct killing effect of the illuminated dye on virus particles.

Knowing the biophysical properties of ACE pigments, I reasoned that if collected ACE pigments were to be activated in close vicinity of herpes virus skin lesions they would still be able to achieve a therapeutic benefit. Such a method would avoid any direct contact of the neutral red or other dye with the herpes skin lesions. This consideration is important since some researchers as well as the US Food and Drug Administration (FDA) have raised the possibility of neutral red inducing oncogenic (cancer causing) mutations in herpes viruses. Moreover, if successful in the therapy of active herpes skin lesions, the approach would likely have broader therapeutic applications to other diseases, including those caused by stealth adapted viruses.

I have further reasoned that ACE-pigments probably exist in four distinct states: i). Directly fluorescent when exposed to UV light; ii) fluorescent only if exposed to UV light after the addition of a small quantity of a fluorescence triggering dye, such as neutral red, to the ACE pigments. The dye seemingly alters ACE pigments in a manner that allows the ACE pigments to better absorb the UV energy and retransmit some of the absorbed energy as fluorescence; iii) non-responsive to either direct or dye enhanced UV illumination because of being non-energy chargeable or vi) non-fluorescent with or without the dye because of being fully charged.

An observation that assisted in this four stage classification was the demonstration that UV activation of ACE pigments in the second of the above stages could achieve conversion of distantly situated second stage pigments to stage one. In other words, neutral red-UV light activation of only a portion of the ACE pigments on a Q-tip can render the remaining ACE pigments on the Q-tip capable of becoming fluorescent when directly placed under UV illumination. Similarly, activation of ACE pigments in one location of the body, for example in a herpes skin lesion can result in partial and more complete systemic activation of the body's ACE pathway. Partial activation is shown when other areas of the patient's skin and/or parts of the patient's oral cavity acquire the property of becoming fluorescent when directly illuminated with UV light. This change commonly accompanies and follows the local UV therapy of neutral red stained herpetic skin lesions. These quite striking observations imply that some form of energy is radiating from the activated stage-two ACE pigments, which were triggered to fluoresce by using neutral red along with UV illumination. This radiated energy is seemingly capable of converting adjacent or even distantly located ACE pigments from stage two to stage one, such that they are now able to fluoresce when directly illuminated with UV light. When fully activated, the ACE pathway will apparently no longer yield ACE pigments that will fluoresce either directly with UV illumination or after the addition of neutral red dye. This is the situation expected in healthy individuals in whom there is no insufficiency of cellular energy.

Prior studies in patients with HSV skin lesions at more than one location in the body have confirmed expedited healing in all lesions even though the neutral red application along with UV illumination was limited to a single lesion. As noted above, although, not necessary for the healing of the distant skin lesions, the ACE pigments in these distant areas will fluoresce if directly examined under UV illumination during the treatment of the lesion selected for neutral red application. Even beyond distant herpes skin lesions, it is usually possible to see UV evoked fluorescence occurring in other areas of the body, such as the soles of the feet and palms of the hands. Furthermore, other skin lesions, such as keratoses, will not uncommonly appear distinct from the surrounding skin because of a heightened fluorescence in response to UV illumination. Inside the mouth is another striking region of the body that can acquire quite marked UV induced fluorescence during and after treating a patient for a localized herpes skin lesion.

Based on these observations, I concluded that a communicating network exists within the body such that it was possible to trigger systemic activation of the ACE pathway by triggering a local response at an area of pathology, or even at a normal appearing area of the body at which ACE pigments have accumulated. An extension of this concept is that even external activation of ACE pigments removed from the body could likely impart a heightened healing capacity of the ACE pathway throughout the body. The heightened healing capacity would be indicated by UV inducible fluorescence at the site of the distinct skin pathology, although the distant healing would not be dependent on the use of UV illumination at the distant site. A further indication of systemic activation of the ACE pathway would be the development of UV inducible fluorescence in other areas of the skin and/or in the oral cavity, etc. With the completion of successful activation of the patient's ACE pathway, it is to be expected that UV and/or dye inducible fluorescence of ACE pigments, either collected from the patient or viewed within skin areas or in the oral cavity of the individual, will no longer occur. This is the situation expected in individual in whom there is no apparent insufficiency of cellular energy.

Based on these considerations, a formal proposal (number 071) entitled “A research study examining the effectiveness of distant activation of ACE pigments associated with recurrent human herpes simplex virus-induced skin lesions” was submitted in 2007 to the Institute of Progressive Medicine, Institutional Review Board (IRB). The proposal received unanimous approval and allowed for the studies that led to the additional observations embodied in this and co-pending patent applications. (References to published articles on Alternative Cellular Energy Pigments (ACE-pigments) and to Stealth Adapted Viruses are listed above. Also included are a series of now abandoned and pending patent applications that provide additional documentation of the earlier findings leading to this present application. Information in all of the published references and cited awarded and abandoned patents applications of William John Martin are incorporated by reference into the present patent application.

### BRIEF SUMMARY OF THE INVENTION

The invention discloses methods and approaches of how to use ACE pigments collected from patients in both the local and systemic activation of the patient's ACE pathway. Activation of the ACE pathway can be useful for the treatment of herpes skin lesions and other viral illnesses, including those caused by stealth adapted viruses. Furthermore, this novel mode of therapy is applicable to an even wider range of illnesses in which there is a presumed insufficiency of cellular energy.

ACE pigments that will fluoresce under UV light illumination after mixing with a small quantity of neutral red dye can be readily collected from patients with various diseases. The sources include active herpes infections as well as from the skin, hair, and body fluids of patients without active skin lesions at the time of collection. The body fluids that can contain ACE pigments include saliva and spittle, urine, and blood (serum, plasma and leukocytes). Collected ACE pigments that are not fully activated are tentatively identified by their UV fluorescence, either directly or more typically when stained with a suitable dye such as neutral red. They are generally particulate in appearance and display light-inducible movements when microscopically examined. They are also generally stainable with iodine/iodide solution. The presence of retrievable UV reactive ACE pigments from a patient is currently being used as a presumptive marker for potential clinical benefit of therapeutic interventions that are aimed at enhancing the patient's ACE pathway. It is not a marker for any particular illness and indeed reactive ACE pigments have been collected from athletes accustomed to strenuous, energy depleting exercises. The optimal goal for both patients and athletes has been to restore non-fluorescence reactivity to UV light of their body's ACE pathway.

The presence of reactive ACE pigments is defined for the purpose of this application as materials generated by the patient that exhibits fluorescence when either directly exposed to UV light or when exposed to UV light after contact with a fluorescence triggering dye such as neutral red. A suitable means of UV illumination is a compact spiral fluorescent light that emits UV-A light. These lights are termed ProLume™ and are available from Halco Lighting (Atlanta, Ga.). The detection of fluorescence can be enhanced by viewing the UV illuminated material in the absence of visible light. The dye that is commonly used to detect ACE pigments is neutral red since it does not fluoresce by itself, yet it readily promotes the fluorescence of reactive ACE pigments. It is also less toxic than some alternative dyes such as acridine orange.

A preferred method for detecting UV reactive ACE pigments from a patient is to stain a Q-tip or other material that has been used to collect ACE pigments from various locations in the body with approximately 0.5 mg/ml solution of neutral red and to look for UV inducible fluorescence. Neutral red can also be directly added to spittle or urine of patients and the mixture illuminated with UV light. Both fluid and dried blood smears can also be stained with neutral red and examined under UV microscopy for intracellular particulate fluorescence. Acridine orange staining is also of diagnostic value when it elicits multicolored fluorescence in stained cells that is distinguishable from the more limited colored fluorescence seen when normal cells are similarly stained.

The present patent application relates specifically to using collected spittle as the source of ACE pigments in the therapy of representative diseases associated with an insufficiency of cellular energy. The two diseases chosen are autism (presumably caused by a stealth adapted virus) and active HSV lesions. These examples reinforce the general principle of using collected ACE pigments in the local and systemic activation of the body's ACE pathway for the purposes of treating diseases

### BRIEF DESCRIPTION OF THE DRAWINGS

None included

### DETAILED DESCRIPTION OF THE INVENTION

Saliva (spittle) was collected from a 14 year old child with autism and shown to contain yellow fluorescing material when mixed with a small quantity of neutral red (approximately 0.1 mg/ml) and illuminated with UV-A light. The child was asked to spit periodically into a container over a several hour period. The goal was to comfortably collect approximately 20 mls of spit. The spit was then placed onto a water dampened kitchen absorbent towel that was then placed onto a sheet of Saran™ wrap that covered the upper back area of the child. The sputum (spittle) towel was then sprayed with a dilute solution of neutral red and illuminated with an UV-A light. The treatment was continued for 45 minutes with occasional re-addition of some saliva to the paper towel followed by additional neutral red spraying. The child was observed for behavioral changes during and after the therapy and quizzed as to the perceived effects of the treatment.

The child chosen for this study has had periodic treatments using the enerceutical™ product “Epione”™ since May of 2008. Both she and her mother were highly reliable research subjects in that they were clearly able to report on the benefits of each therapy session. Moreover, they correctly identified various additional test batches of materials that were subsequently shown to lack the enerceutical™ activity of Epione.™ The question posed to the child and to her mother was whether the spittle protocol was comparable in its benefits to that of active Epione.™ The answer was very much in the affirmative such that even two weeks later, the child requested the use of her spit rather than Epione.™ In this session, the spittle moistened towels were placed onver the popliteal fossa (back of the knees). Other suitable sites include the palms of the hands, soles of the feet and the face (with the patient wearing UV barrier goggles.

The child's mother provided a detailed description of the benefits seen after both the first and the second treatments. Specifically, the child feels a pleasurable systemic change during the therapy session. Almost immediately following the treatment and readily seen on the next day, she exhibits a major heightening of her level of self-awareness. She becomes more articulate and even humorous in her conversations, performs far better at speech therapy and is more attentive and considerate of others. For at least a week after a successful therapy session, she rarely has any of the absence seizures that were very common prior to beginning therapy in May 2008 and which still begin to slowly reoccur a week or so following therapy. The mother is convinced that the benefit from using the spit is comparable to that seen when using active formulations of Epione and far better that the lack of any discernable benefit from using control materials. The mother is also regularly monitoring the intensity of the fluorescence seen when her child's spit is mixed with neutral red and illuminated with a UV light. Although not yet definitive and at no time has the fluorescence disappeared, it is thought that the intensity does vary with the wellness of the child.

In a similar sequence of events, a patient with an active HSV lesion on her upper lip was asked to test her saliva for neutral red inducible UV fluorescence. She had been asked to do so earlier in response to informing me that she was prone to HSV outbreaks at times of stress or concomitant illnesses. While a low level of spit fluorescence was seen when the asymptomatic patient was initially tested, the level of fluorescence was much more marked when the HSV lesion had developed. For the initial testing, the patient was asked to collect saliva onto a Q-tip, stain the Q-tip with neutral red and place it onto a small piece of Saran™ wrap laid against the HSV skin lesion. When illuminated with a UV light, the Q-tip brightly fluoresced as did the underlying HSV lesion and surrounding skin area Holding Q-tip against the HSV lesion was causing some discomfort due to pressure and was tiresome on the arm. The procedure was, therefore, quickly modified to moisten a small piece of paper with saliva, stain with neutral red and simply lay it onto the lesion with an intervening layer of Saran™ wrap. In a darkened room, she could see the bright fluorescence on the paper and on the underlying lesion. She also noted the swelling and tingling of the skin lesion along with a reduction of its pain. By the next day, the lesion had largely healed something that would have ordinarily taken 4-5 days.

The spittle or saliva protocol has been offered as an alternative to the use of Epione™ in other patients, including a patient with shingles caused by herpes zoster virus (HZV). Although Saran™ wrap is transparent; the procedure does not involve the transfer of light energy between the paper and the skin, since a light impermeable layer can be used instead of the Saran™ wrap. An advantage of a transparent layer is that by simply peeling back the paper layer, one can periodically observe any underlying lesion for UV inducible fluorescence. The present hypothesis is that there is a coupling of the energy from the neutral red stained ACE pigments on the paper towel to unstained ACE pigments in the skin. Absorption of this energy converts the body's ACE pigments from stage 2 (neutral red dependent fluorescence) to stage 1 (directly fluorescent) and then onto stage 4 (fully charged). The nature of the transferred energy(s) is under active investigation.

The spittle or saliva protocol is a practical alternative to collecting ACE pigments from active HSV, HZV and other accessible viral induced skin lesions. Probing a herpetic skin lesion can be painful and it can be difficult to collect material from a lesion once it is beginning to scab. While general areas of the skin and hair can provide ACE pigments in these and other types of disease conditions, the distribution of ACE pigments can be quite irregular in contrast to the collection of saliva. Moreover, assessing the neutral red inducible fluorescence of saliva can be a useful way of monitoring the ACE pathway in a patient.

The use of spittle is not intended as a replacement of Epione™ or other suitable enerceutical™ products under most situations. Rather it simply provides a lower cost alternative. It is fully anticipated that spittle from one person may be used to activate the ACE pathway in some other individuals and that a direct method of ACE activating energy transfer into humans and animals will soon be developed.

Various modifications and adaptations of this basic approach of using ACE pigments collected from saliva to achieve overall and even local activation of the body's ACE pathway will be apparent to any practitioner who has followed this line of scientific inquiry. These include the use of a wide range of collection materials, including urine, blood, effusions; other dyes; other UV light sources; etc. Furthermore, the medical conditions that can potentially be treated using the method of externally activating the ACE pigments obtained from a patient, or even from another individual, are not confined to autism or HSV skin lesions. A more basic understanding of the utility of the described ACE activation method relates to the broader concept that many individuals have an insufficiency of cellular energy to fully cope with disease processes. In one context, the ACE pathway is viewed as an adjunct to the immune system in combating various types of conventional infections, including those due to herpes viruses, papillomaviruses, hepatitis viruses, influenza, etc. The ACE pathway is even more critically involved in the defense of infections caused by stealth adapted viruses that are not effectively recognized by the cellular immune system. Similarly, the ACE pathway can potentially play an increasingly important role in the evolving immune suppression caused by infections that directly target the immune system such as HIV or in extremely rapid infections that outpace the development of an effective immune response, such as Ebola.

Autism is viewed as being part of a wide spectrum of illnesses associated with stealth adapted viruses. Approximately a third of autistic children show evidence of epilepsy and ongoing clinical studies have confirmed a reduction in seizure activity upon administering ACE therapy to autistic children. ACE therapy has been successfully employed in treating patients with psychiatric and neurological illnesses. Some of these illnesses can also be associated with persisting infections with stealth adapted viruses. Certain disease entities, including infections with stealth adapted viruses, can be associated with the production of relatively large amounts of ACE pigments. Indeed, many of the pigments will fluorescence when directly exposed to UV light, although usually not nearly as much as when mixed with neutral red or some other dyes. The most prominent of these disease entities is currently being referred to as delusional parasitosis or Morgellon's disease. The strong electrostatic and other energy converting capacity of the ACE pigments has led some observers to the mistaken belief that they are living parasites. The common psychiatric component of this illness explains why their physicians attribute such beliefs to delusions.

Non-infectious diseases for which the level of ACE pathway activation may play a crucial role include emphysema and other respiratory diseases in which the tissues receive insufficient oxygen for normal mitochondria based metabolism. Similarly, cellular metabolism can be restricted in the brain and heart by cerebrovascular and cardiovascular diseases, respectively. Cancer is yet another disease in which a cellular energy deficiency can be posited as limiting a healing process. The option clearly exists for testing the ACE pathway in virtually all forms of diseases and augmenting it using either the body's own ACE pigments as illustrated in this patent application or formulated sources as described in previous and co-pending patent applications.

While the major focus of prior and present studies on the ACE pathway is on humans, it is clear that the methods under study are also applicable to infectious and other insufficiency of cellular energy associated disease states in animals. The ACE pathway is also considered to be operative in plants and various plant derived materials, included in the broader category of ACE-pigments related materials that I have termed Enerceuticals.™

An important characteristic of Enerceuticals is that they do not need to localize to the site of disease pathology since they can exert a “field effect” applicable over a distance. Hence it is possible to use energy stimulated ACE pigments external to the body to activate the body's ACE pathway and to achieve therapeutic benefits throughout the body. Note also that ultraviolet light is not the only potential source of energy that can activate ACE pigments collected from a patient and placed in very close proximity to the patient's skin, or even within one or other of the patient's cavities or orifices.

Various devices and kits are envisioned that will facilitate the placement of the spittle/dye mixture onto discrete skin lesions or other areas of the body. A slightly adhesive quality would be useful for covering lesions that are not in a horizontally supported position. A central window in the absorbent material could help in viewing an underlying lesion for fluorescence. A squeezable open sputum collection bottle that could be subsequently fitted with a spray head or other dispensing nozzle could be designed and potentially pre-filled with a small quantity of a mucolytic agent.

The sun can also be used as an alternative source of ultraviolet light rather than relying on a light bulb and source of electricity. Even viewing the saliva for dye inducible or direct fluorescence can be done in daylight using a glass or plastic filter that allows selective passage of UV light into a dark box in which the sputum±dye, either alone or attached to a suitable carrier; such as a Q-tip, added.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Additional advantages and modifications will readily occur to those skilled in the art. Variations and changes may be made without departing from the spirit of the invention encompassed by the appended claims.

What I claim as my invention is:

1. A method of activating the alternative cellular energy (ACE) pathway in a human or animal subject comprising the collection of saliva (spittle) from the patient;
adding a quantity of dye, such as neutral red or acridine orange, to the ACE pigments contained within the spittle so as to render them fluorescent or otherwise activated when illuminated with a source of ultraviolet (UV) lighting; and placing the mixture of activated ACE pigments and dye onto a surface area of the body and continuing with the ultraviolet illumination in a manner that will activate the ACE pathway in the body and achieve a therapeutic benefit within the subject.

2. The method of claim 1 in which the illness being treated is autism.

3. The method of claim 1 in which the condition being treated is a mental illness.

4. The method of claim 1 in which the illness is a neurological disease.

5. The method of claim 1 in which the condition is a respiratory, cardiovascular or cerebovascular disease associated with an insufficiency of cellular energy.

6. The method of claim 1 in which the condition is a behavioral and/or learning disorder affecting a child or an adult.

7. The method of claim 1 in which the illness is the chronic fatigue syndrome and/or fibromyalgia.

8. The method of claim 1 in which the condition is referred to as delusional parasitosis or Morgellon's disease

9. The method of claim 1 in which the illness being treated is epilepsy

10. The method of claim 1 in which the condition being treated is an infectious disease.

11. The method of claim 1 in which the condition being treated is caused by active herpes simplex virus or herpes zoster virus.

12. The method of claim 1 in which the person being treated experiences recurrent outbreaks of herpes infections, yet no active herpes infection is present at the time of treatment.

13. The method of claim 1 in which are person experiences symptoms resulting from a prior outbreak of active herpes zoster virus infection but without any active lesions being present at the time of treatment.

14. The method of claim 1 in which the dye used is neutral red at a concentration of between 0.05 to 5.0 milligrams per ml.

15. The method of claim 1 in which the dye used is acridine orange at a concentration of between 0.05 to 5.0 milligrams per ml.

16. The method of claim 1 in which the source of ultraviolet light is a lamp of between 5 and 200 watts that emits at a maximum wavelength between 350 and 380 nanometers.

17. The method of claim 1 in which sunlight is used as the source of ultraviolet illumination.

18. The method of claim 1 in which a filter is used that allows for the passage of ultraviolet light into a darkened area within which one can observe fluorescence.

19. The method of claim 1 that comprises a kit on which to place the salivadye mixture for placement onto the body.

20. The method of claim 1 that comprises a container for collecting saliva and which may contain a mucolytic agent to diminish the viscosity of the saliva and allow for easier application to absorbent material, such as paper or cloth.
